(19) United States
(12) Patent Application Publication
(10) Pub. No.: US 2006/0040937 A1
(43) Pub. Date: Feb. 23, 2006

(54) N-aroyl cyclic amines as orexin receptor antagonists

(75) Inventors: Clive Lesile Branch, King of Prussia, PA (US); Steven Coulton, Harlow Essex (GB); Amanda Johns, Harlow (GB); David John Nash, Harlow (GB); Roderick Alan Porter, Harlow (DE); Geoffrey Stemp, Harlow (DE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited, Greenford, Middlesex (GB)

(21) Appl. No.: 10/527,833

(22) PCT Filed: Sep. 17, 2003
(86) PCT No.: PCT/EP03/10412

§ 371 Date: Aug. 16, 2005

(30) Foreign Application Priority Data
Sep. 18, 2002 (GB) 0221691.9
Sep. 18, 2002 (GB) 0221690.1

(51) Int. Cl. A61K 31/5377; A61K 31/506; A61K 31/496; C07D 413/02; C07D 403/02

(52) U.S. Cl. 514/235.5; 514/252.14; 514/253.01; 544/120; 544/122; 544/295; 544/360

(57) ABSTRACT

This invention relates to N-aroyl cyclic amine derivatives and their use as pharmaceuticals.

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP-A-875565, EP-A-875566 and WO 96/34877. Polypeptides and polynucleotides encoding a second human orexin receptor, orexin-2 (HFGANP), have been identified and are disclosed in EP-A-893498.

Polypeptides and polynucleotides encoding polypeptides which are ligands for the orexin-1 receptor, e.g. orexin-A (Lig72A) are disclosed in EP-A-849361.

Orexin receptors are found in the mammalian host and may be responsible for many biological functions, including pathologies including, but not limited to, depression; anxiety; addictions; obsessive compulsive disorder; affective neurosis/disorder; depressive neurosis/disorder; anxiety neurosis; dysthymic disorder; behaviour disorder; mood disorder; sexual dysfunction; psychosexual dysfunction; sex disorder; sexual disorder; schizophrenia; manic depression; delerium; dementia; severe mental retardation and dyslinesias such as Huntington's disease and Gilles de la Tourett's syndrome; disturbed biological and circadian rhythms; feeding disorders, such as anorexia, bulimia, cachexia, and obesity; diabetes; appetite/taste disorders; vomiting/nausea; asthma; cancer; Parkinson's disease; Cushing's syndrome/disease; basophil adenoma; prolactinoma; hyperprolactinemia; hypopituitarism; hypophysis tumor/adenoma; hypothalamic diseases; Froehlich's syndrome; adrenohypophysis disease; bypophysis disease; hypophysis tumor/adenoma; pituitary growth hormone; adrenohypophysis hypofunction; adrenohypophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth hormone deficiency; dwarfism; gigantism; acromegaly; disturbed biological and circadian rhythms; and sleep disturbances associated with such diseases as neurological disorders, neuropathic pain and restless leg syndrome, heart and lung diseases; acute and congestive heart failure; hypotension; hypertension; urinary retention; osteoporosis; angina pectoris; myocardial infarction; ischaemic or haemorrhagic stroke; subarachnoid haemorrhage; head injury such as sub-arachnoid haemorrhage associated with traumatic head injury; ulcers; allergies; benign prostatic hypertrophy; chronic renal failure; renal disease; impaired glucose tolerance; migraine; hyperalgesia; pain; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to infection, e.g. HIV, post-polio syndrome, and post-herpetic neuralgia; phantom limb pain; labour pain; cancer pain; post-chemotherapy pain; post-stroke pain; post-operative pain; neuralgia; nausea and vomiting; conditions associated with visceral pain including irritable bowel syndrome, migraine and angina; urinary bladder incontinence e.g. urge incontinence; tolerance to narcotics or withdrawal from narcotics; sleep disorders; sleep apnea; narcolepsy; insomnia; parasomnia; jet-lag syndrome; and neurodegenerative disorders, which includes nosological entities such as disinhibition-dementia-parkinsonism-amyotrophy complex; pallido-ponto-nigral degeneration, epilepsy, and seizure disorders.

Experiments have shown that central administration of the ligand orexin-A (described in more detail below) stimulated food intake in freely-feeding rats during a 4 hour time period. This increase was approximately four-fold over control rats receiving vehicle. These data suggest that orexin-A may be an endogenous regulator of appetite. Therefore, antagonists of its receptor may be useful in the treatment of obesity and diabetes, see Cell, 1998, 92, 573-585.

There is a significant incidence of obesity in westernised societies. According to WHO definitions a mean of 35% of subjects in 39 studies were overweight and a further 22% clinically obese. It has been estimated that 5.7% of all healthcare costs in the USA are a consequence of obesity. About 85% of Type 2 diabetics are obese, and diet and exercise are of value in all diabetics. The incidence of diagnosed diabetes in westemised countries is typically 5% and there are estimated to be an equal number undiagnosed. The incidence of both diseases is rising, demonstrating the inadequacy of current treatments which may be either ineffective or have toxicity risks including cardiovascular effects. Treatment of diabetes with sulfonylureas or insulin can cause hypoglycaemia, whilst metformin causes GI side-effects. No drug treatment for Type 2 diabetes has been shown to reduce the long-term complications of the disease. Insulin sensitisers will be useful for many diabetics, however they do not have an anti-obesity effect.

Rat sleep/EEG studies have also shown that central administration of orexin-A, an agonist of the orexin receptors, causes a dose-related increase in arousal, largely at the expense of a reduction in paradoxical sleep and slow wave sleep 2, when administered at the onset of the normal sleep period. Therefore antagonists of its receptor may be useful in the treatment of sleep disorders including insomnia.

The present invention provides N-aroyl cyclic amine derivatives which are non-peptide antagonists of human orexin receptors, in particular orexin-l receptors. In particular, these compounds are of potential use in the treatment of obesity, including obesity observed in Type 2 (non-insulin-dependent) diabetes patients, and/or sleep disorders. Additionally these compounds are useful in the treatment of stroke, particularly ischemic or haemorrhagic stroke, and/or blocking the emetic response, i.e. useful in the treatment of nausea and vomiting.

International Patent Applications WO99/09024, WO99/58533, WO00/47577 and WO00/47580 disclose phenyl urea derivatives and WO00/47576 discloses quinolinyl cinnamide derivatives as orexin receptor antagonists. WO01/96302 discloses N-aroyl cyclic amine derivatives.

According to the invention there is provided a compound of formula (1): 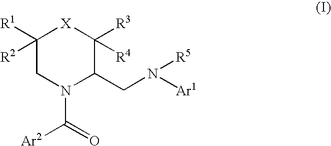
wherein:

X is O, CR⁷R⁸, NH or bond;

R¹ and R² are both hydrogen, both optionally substituted (C₁₋₄)alkyl, or are together with the carbon to which they are attached form a (C₃₋₆)cycloalkyl ring or a 4- to 6-membered heterocyclyl ring.

R³ and R⁴ are both hydrogen, both optionally substituted (C₁₋₄) alkyl, or are together with the carbon to which they are attached form a (C₃₋₆)cycloalkyl ring or a 4- to 6-membered heterocyclyl ring;

R⁷ and R⁸ are both hydrogen, both optionally substituted (C₁₋₄) alkyl, or are together with the carbon to which they are attached form a (C₃₋₆)cycloalkyl ring or a 4- to 6-membered heterocyclyl ring;

provided that one pair of R¹ and R², R³ and R⁴, R⁷ and R⁸ are both optionally substituted (C₁₋₄)alkyl, or are together with the carbon to which they are attached form a (C₃₋₆)cycloalkyl ring or a 4- to 6-membered heterocyclyl ring and the remaining groups are hydrogen;

R⁵ is hydrogen, optionally substituted (C₁₋₄)alkyl, or optionally substituted (C₁₋₄)alkylCO;

Ar¹ is an optionally substituted aryl, an optionally substituted mono or bicyclic heteroaryl group containing up to 3 heteroatoms selected from N, O and S;

Ar² represents phenyl or a 5- or 6-membered heterocyclyl group containing up to 3 heteroatoms selected from N, O and S, wherein the phenyl or heterocyclyl group is substituted by R⁶ and further optional substituents; or Ar² represents an optionally substituted bicyclic aromatic or bicyclic heteroaromatic group containing up to 4 heteroatoms selected from N, O and S;

R⁶ represents hydrogen, optionally substituted(C₁₋₄ )alkoxy, halo, cyano, optionally substituted(C₁₋₆)alkyl, optionally substituted phenyl, or an optionally substituted 5- or 6-membered heterocyclyl group containing up to 4 heteroatoms selected from N, O and S;

or a pharmaceutically acceptable salt thereof.

When R¹ and R², R³ and R⁴ or R⁷ and R⁸ are optionally substituted (C₁₋₄)alkyl, the optionally substituted alkyl groups can be the same or different.

Preferably R⁷ and R⁸ if present are hydrogen.

Preferably R¹, R², R⁷ and R⁸ are hydrogen when R³ and R⁴ are methyl or R³, R⁴, R⁷ and R⁸ are hydrogen when R¹ and R² are methyl.

Preferably X is CR⁷R⁸

Preferably R⁵ is hydrogen or optionally substituted (C₁₋₄)alkyl, more preferably hydrogen.

Preferably where Ar² represents phenyl or a 5- or 6-membered heterocyclyl group containing up to 3 heteroatoms selected from N, O and S, the R⁶ group is situated adjacent to the point of attachment to the amide carbonyl.

Ar¹ may have up to 5, preferably 1, 2 or 3 optional substituents.

Examples of when Ar¹ is a mono or bicyclic heteroaryl group are quinoxalinyl, quinazolinyl, pyridopyrazinyl, benzoxazolyl, benzothiophenyl, benzimidazolyl, naphthyridinyl, pyridinyl, pyrimidinyl, thiazolyl, pyridazinyl, pyrazinyl, oxazolyl, triazolyl, imidazolyl, pyrazolyl, quinolinyl, benzofuranyl, indolyl, benzothiazolyl, oxazolyl[4,5-b]pyridinyl, pyridopyrimidinyl, isoquinolinyl, furanyl or thienyl.

Preferably Ar¹ is pyrimidinyl or pyridinyl.

When Ar² is a 5- or 6-membered heterocyclyl group containing up to 3 heteroatoms selected from N, O and S, it may be furanyl, thienyl, pyrrolyl, oxazolyl, thiazolyl, imidazolyl, oxadiazolyl, thiadiazolyl, pyridinyl, triazolyl, triazinyl, pyridazinyl, pyrimidinyl, isothiazolyl, isoxazolyl, pyrazinyl or pyrazolyl.

When Ar² is an optionally substituted bicyclic aromatic or heteroaromatic it may be selected from benzofuranyl, benzimidazolyl, quinolinyl, quinoxalinyl, naphthyl, benzotriazolyl, benzothienyl, benzoxazolyl, naphthyridinyl, isoquinolinyl, quinazolinyl, indolyl, benzothiazolyl, or benzothiadiazolyl.

Preferably Ar² represents optionally substituted quinolinyl, thiazolyl, pyrazolyl, phenyl, naphthyl or quinoxalinyl.

When R⁶ is a 5- or 6-membered heterocyclyl group containing up to 4 heteroatoms selected from N, O and S, it may be furanyl, thienyl, pyrrolyl, oxazolyl, thiazolyl, imidazolyl, oxadiazolyl, thiadiazolyl, pyridinyl, triazolyl, triazinyl, pyridazinyl, pyrimidinyl, isothiazolyl, isoxazolyl, pyrazinyl, pyrazolyl, tetrazolyl, piperazinyl, piperidinyl, morpholinyl, thiomorpholinyl or pyrrolindinyl.

Preferably when R⁶ is a 5- or 6-membered heterocyclic ring containing up to 4 heteroatoms selected from N, O and S, it may be furanyl, thienyl, pyrrolyl, oxazolyl, thiazolyl, imidazolyl, oxadiazolyl, thiadiazolyl, pyridinyl, triazolyl, triazinyl, pyridazinyl, pyrimidinyl, isothiazolyl, isoxazolyl, pyrazinyl or pyrazolyl.

Preferably R⁶ is selected from trifluoromethoxy, methoxy, ethoxy, halo, or an optionally substituted phenyl, pyridinyl, pyrazolyl, pyrimidinyl, or oxadiazolyl group.

When R¹ and R² or R³ and R⁴ or R⁷ and R⁸ together with the carbon to which they are attached form a 4- to 6-membered heterocyclyl ring, it may be selected from morpholinyl, piperidinyl, pyrrolidinyl, tetrahydrofuranyl, oxetanyl or azetidinyl.

Optional substituents for the groups Ar¹, Ar², and R⁶ include halogen, hydroxy, oxo, cyano, nitro, (C₁₋₄)alkyl, (C₁₋₄)alkoxy, hydroxy(C₁₋₄)alkyl, hydroxy(C₁₋₄)alkoxy, halo(C₁₋₄)alkyl, halo(C₁₋₄)alkoxy, aryl(C₁₋₄)alkoxy, (C₁₋₄)alkylthio, hydroxy(C₁₋₄)alkyl, (C₁₋₄)alkoxy(C₁₋₄)alkyl, (C₃₋₆)cycloalkyl(C₁₋₄)alkoxy, (C₁₋₄)alkanoyl, (C₁₋₄)alkoxycarbonyl, (C₁₋₄)alkylsulfonyl, (C₁₋₄)alkylsulfonyloxy, (C₁₋₄)alkylsulfonyl(C₁₋₄)alkyl, arylsulfonyl, arylsulfonyloxy, arylsulfonyl(C₁₋₄)alkyl, (C₁₋₄)alkylsulfonamido, (C₁₋₄)alkylamido, (C₁₋₄)alkylsulfonamido(C₁₋₄)alkyl, (C₁₋₄)alkylamido(C₁₋₄)alkyl, arylsulfonamido, arylcarboxamido, arylsulfonamido(C₁₋₄)alkyl, arylcarboxamido(C₁₋₄)alkyl, aroyl, aroyl(C₁₋₄)alkyl, or aryl(C₁₋₄)alkanoyl group; a group R^{a}R^{b}N—, R^{a}OCO(CH₂)ᵣ, R^{a}CON(R^{a})(CH₂)ᵣ, R^{a}R^{b}NCO(CH₂)ᵣ, R^{a}R^{b}NSO₂(CH₂)ᵣ or R^{a}SO₂NR^{b}(CH₂), where each of R^{a} and R^{b} independently represents a hydrogen atom or a (C₁₋₄)alkyl group or where appropriate R^{a}R^{b} forms part of a (C₃₋₆)azacyloalkane or (C₃₋₆)(2-oxo)azacycloalkane ring and r represents zero or an integer from 1 to 4, (C₁₋₄)acyl, aryl, aryl(C₁₋₄)alkyl, (C₁₋₄)alkylamino(C₁₋₄)alkyl, R^{a}R^{b}N(CH₂)n-, R^{a}R^{b}N(CH₂)nO-, wherein n represents an integer from 1 to 4, or when the substituent is R^{a}R^{b}N(CH₂)n- or R^{a}R^{b}N(CH₂)nO, R^{a} with at least one CH₂ of the (CH₂)n portion of the group form a (C₃₋₆)azacycloalkane and R^{b} represents hydrogen, a (C₁₋₄)alkyl group or with the nitrogen to which it is attached forms a second (C₃₋₆)azacycloalkane fused to the first (C₃₋)azacycloalkane.

Preferred optional substituents for Ar² are halogen, cyano, (C₁₋₄)alkyl, or (C₁₋₄)alkoxy.

Preferred optional substituents for Ar¹ are halogen. Most preferably the optional substituent for Ar¹ is bromine.

Preferred optional substituents for R⁶ are halogen.

When R¹ to R⁴, R⁷ and R⁸ are (C₁₋₄) alkyl the optionally substitutents can be halogen, hydroxy, (C₁₋₄)alkoxy, hydroxy(C₁₋₄)alkyl, hydroxy(C₁₋₄)alkoxy, (C₁₋₄)alkylthio, hydroxy(C₁₋₄)alkyl, (C₁₋₄)alkoxy(C₁₋₄)alkyl or (C₃₋₆)cycloalkyl(C₁₋₄)alkoxy.

In the groups Ar¹ and Ar², substituents positioned ortho to one another may be linked to form a ring.

Preferred compounds of formula (I) are selected from:
(RS)-1-{2-[(5-Bromo-pyrimidin-2-ylamino)-methyl]-3,3-dimethyl-piperidin-1-yl}-1-[5-(4-fluoro-phenyl)-2-methyl-thiazol-4-yl]-methanone;
(RS)-1-{2-[(5-Bromo-pyrimidin-2-ylamino)-methyl]-3,3-dimethyl-piperidin-1-yl}-1-quinolin-8-yl-methanone;
1-{2-[(5-Bromo-pyrimidin-2-ylamino)-methyl]-3,3-dimethyl-piperidin-1-yl}-1-(2-methyl-quinolin-5-yl)-methanone;
(RS)-1-{2-[(5-Bromo-pyrimidin-2-ylamino)-methyl]-5,5-dimethyl-piperidin-1-yl}-1-(2-methyl-quinolin-5-yl)-methanone;
1-{2-[(5-Bromo-pyrimidin-2-ylamino)-methyl]-5,5-dimethyl-piperidin-1-yl}-1-(2-methyl-quinolin-5-yl)-methanone;
(RS)-1-{2-[(5-Bromo-pyridin-2-ylamino)-methyl]-3,3-dimethyl-piperidin-1-yl}-1-(2,3-dimethyl-quinoxalin-5-yl)-methanone;
(RS)-1-{2-[(5-Bromo-pyridin-2-ylamino)-methyl]-3,3-dimethyl-piperidin-1-yl}-1-(2-methyl-quinolin-5-yl)-methanone; or
1-{(S)-2-[(5-Bromo-pyrimidin-2-ylamino)-methyl]-3,3-dimethyl-piperidin-1-yl}-1-(2,3-dimethyl-quinolin-8-yl)-methanone;
and pharmaceutically acceptable salts thereof.

When a halogen atom is present in the compound of formula (I) it may be fluorine, chlorine, bromine or iodine.

When the compound of formula (I) contains an alkyl group, whether alone or forming part of a larger group, e.g. alkoxy or alkylthio, the alkyl group may be straight chain, or branched or combinations thereof, it is preferably methyl or ethyl.

When used herein the term (C₄₋₆)cycloalkyl means a cycloalkyl group having 4, 5 or 6 carbon atoms, for instance cyclopropyl, cyclobutyl or cyclohexyl. Preferably it is cyclopropyl. Cycloalkyl groups can additionally be substituted by straight or branched alkyl groups.

When used herein the term aryl means a 5- to 6-membered aromatic ring for example phenyl, or a 7 to 12 membered bicyclic ring system where at least one of the rings is aromatic for example naphthyl.

It will be appreciated that compounds of formula (I) may exist as R or S enantiomers. The present invention includes within its scope all such isomers, including mixtures. Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

It will be understood that the invention includes pharmaceutically acceptable derivatives of compounds of formula (I) and that these are included within the scope of the invention.

Particular compounds according to the invention include those mentioned in the examples and their pharmaceutically acceptable derivatives.

As used herein “pharmaceutically acceptable derivative” includes any pharmaceutically acceptable salt, ester or salt of such ester of a compound of formula (I) which, upon administration to the recipient is capable of providing (directly or indirectly) a compound of formula (I) or an active metabolite or residue thereof.

It will be appreciated that for use in medicine the salts of the compounds of formula (I) should be pharmaceutically acceptable. Suitable pharmaceutically acceptable salts will be apparent to those skilled in the art and include acid addition salts formed with inorganic acids e.g. hydrochloric, hydrobromic, sulphuric, nitric or phosphoric acid; and organic acids e.g. succinic, maleic, acetic, fumaric, citric, tartaric, benzoic, p-toluenesulfonic, methanesulfonic or naphthalenesulfonic acid. Other salts e.g. oxalates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further feature of the invention there is provided a process for the preparation of compounds of formula (I) and derivatives thereof. The following schemes detail some synthetic routes to compounds of the invention.
Scheme 1 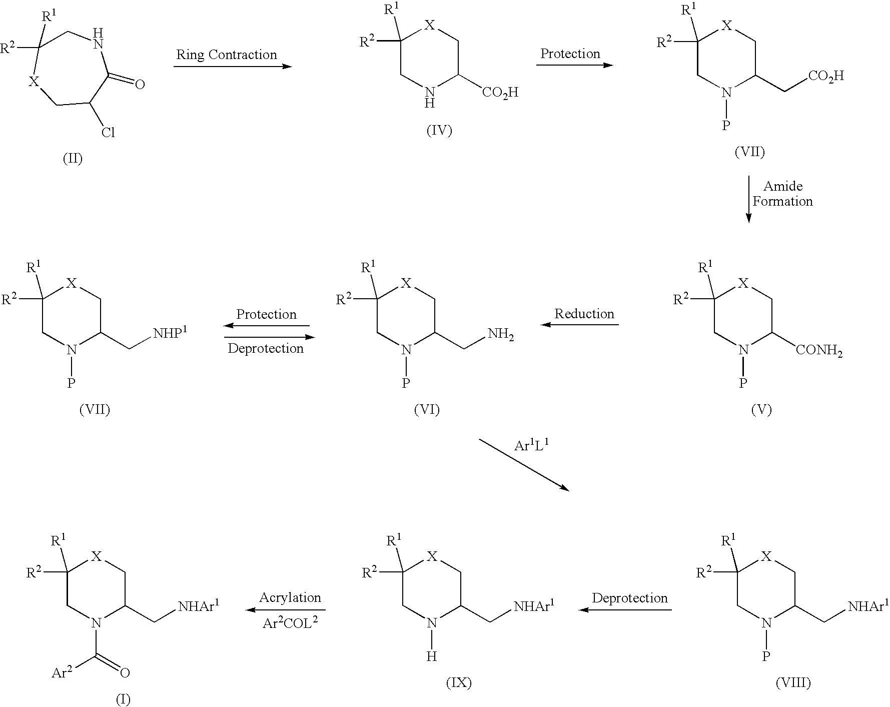
wherein X, Ar¹ and Ar² are as defined for formula (I), R¹ and R² are both optionally substituted (C₁₋₄)alkyl, or are together with the carbon to which they are attached form a (C₃₋₆)cycloalkyl ring or a 4- to 6-membered heterocyclyl ring, L¹ and L² are leaving groups, P and P¹ are protecting groups.

Examples of suitable leaving groups L¹ include halogen, OSO₂Me and OSO₂CF₃. Reaction of amine (VI) to afford amine (VIII) proceeds in an inert solvent such as xylene, dimethylformamide, N-methylpyrrolidinone or a hydroxylic solvent such as t-butanol, in the presence of a base such as potassium carbonate, or diisopropylethylamine, preferably at elevated temperatures.

Examples of leaving groups L² include halogen, hydroxy, OC(═O)alkyl, OC(═O)O-alkyl and OSO₂Me. Acylation may be carried out using a wide range of conditions known in the literature, e.g. in an inert solvent such as dichloromethane, in the presence of a base such as triethylamine. Alternatively these steps may be carried out when L² represents hydroxy, in which case the reaction takes place in an inert solvent such as dichloromethane in the presence of a diimide reagent such as 1-ethyl-3-(3-dimethylaminopropyl)carbodiimide hydrochloride, and an activator such as 1-hydroxybenzotriazole or in dimethylformamide with O-(7-azabenzotriazol-1-yl)-N,N,N′,N′-tetramethyluronium hexafluorophosphate

Examples of protecting groups P and P¹ include t-butyloxycarbonyl, trifluoroacetyl, benzyloxycarbonyl and optionally substituted benzyl. Deprotection conditions will depend on the particular protecting group; for the groups mentioned above these are respectively, acid (e.g. trifluoroacetic acid in dichloromethane), base (e.g. potassium carbonate in a solvent such as aqueous methanol) and catalytic hydrogenolysis in an inert solvent (e.g. using palladium on charcoal in a lower alcohol or ethyl acetate).

Compounds of formula (II) are known in the literature or can be prepared by known methods and converted into acids of type (II) using methods known in the art. For example, when R¹ and R² are both Me, and X is CH₂, according to EP 0447704 A1.

Reduction of the amide (V) to amine (VI) can be achieved using known methods e.g. by use of a metal hydride such as lithium aluminium hydride or borane, in an inert solvent such as tetrahydrofuran or diethyl ether.

Alkylation of compounds of formula (VII) to produce compounds where R⁵ is optionally substituted alkyl, can be achieved using known methods e.g. by use of an alkylating agent such as methyl iodide in the presence of a metal hydride such as sodium hydride in a solvent such as dimethylformamide.

Compounds of formula (I) wherein R⁵ is optionally substituted (C₁₋₄)alkylCO can be made from compounds of formula (I) wherein R⁵ is hydrogen by acylation reaction known in the literature.

Within the scope of this scheme, conversion of amine (VI) to amine (VIII) by reaction with a group Ar¹L¹ can also be achieved without a protecting group P i.e. in compounds (VI) and (VIII) P can be H.

Included within the scope is protecting group interchange and use of optional protecting groups within Ar¹, Ar², R¹, R², R³, R⁴ and X for example when X is NH, preferably a protecting group is used.

The synthetic route outlined in Scheme 1 can also be used for compounds (I) where R¹ and R² are H and X is CH₂, and R³ and R⁴ are as defined for formula (I), from compounds of formula (X) and (XI) which can be synthesised by methods known in the literature. For example when R³ and R⁴ are both Me, and X is CH₂ according to EP 0447704 A1 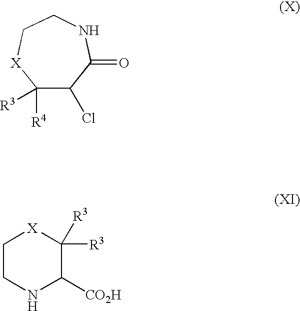
or where R⁷ and R⁸ are both Me, according to EP 0447704 A1 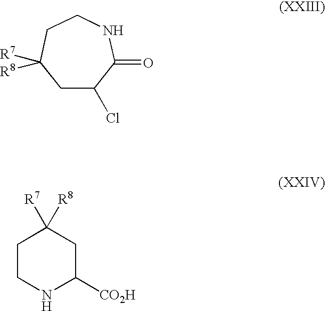
Compounds of formula (XXIV) wherein R⁷ and R⁸ and the NH group is protected by a Boc group are disclosed in Castro et al, J. Med. Chem 1997, 40, 2491-2501.

Where X is a bond, compounds of the following formula are disclosed as follows: 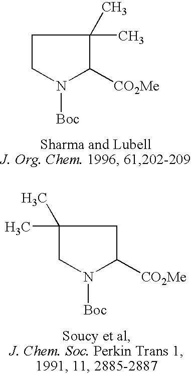 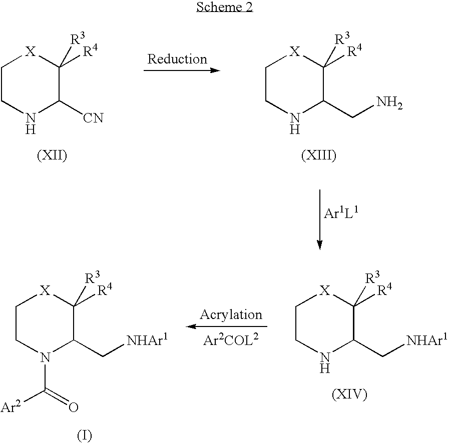
wherein X, Ar¹ and Ar² are as defined for formula (I), R³ and R⁴ are both optionally substituted (C₁₋₄)alkyl, or are together with the carbon to which they are attached form a (C₃₋₆)cycloalkyl ring or a 4- to 6-membered heterocyclyl ring and L¹ and L² are leaving groups and P¹ and P² are protecting groups as defined in Scheme 1.

Reduction of the nitrile (XII) to amine (XIII) can be achieved using known methods e.g. by the use of a metal hydride such as lithium aluminium hydride in an inert solvent such as tetrahydrofuran. Conversion of intermediates (XIII) to (XIV) and product (I) can be achieved as described for Scheme 1.

Compounds of formula (XII) can be synthesised using methods known in the literature. For example, when R³ and R⁴ are both Me and X is CH₂ , according to Martens et al. J. Chem. Soc. Perkin Trans 1, 2001, 508-13.

Included within the scope is protecting group interchange and use of optional protecting groups within Ar¹, Ar², R¹, R², R³, R⁴ and X for example when X is NH, preferably a protecting group is used. 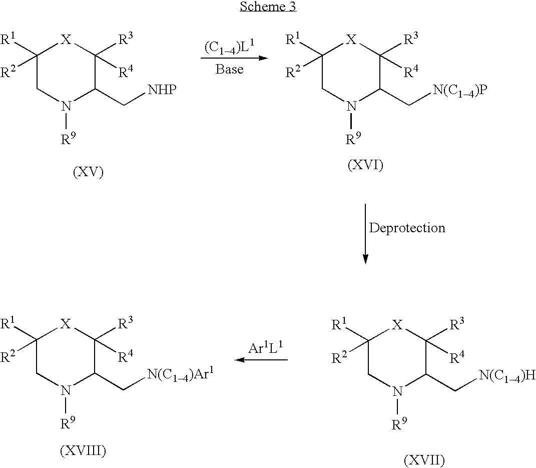
wherein R¹, R², R³, R⁴, X and Ar¹ are as defined for formula (I) and L¹ is a leaving group and P a protecting group as defined for Scheme 1. R⁹ can be either a protecting group P¹ or Ar²CO as defined for Scheme 1 and formula (I) respectively. For the conversion of (XVII) to (XVIII) R⁹ can be H. In compounds of formula (XVIII) when R⁹ is a protecting group P¹, deprotection gives (XVIII, R⁹═H). Acylation of (XVIII) (R⁹═H) with a group Ar²COL² affords compounds of formula (I).

Reaction of (XV) with an alkylating agent (C₁₋₄)L¹ proceeds in the presence of a base such as sodium hydride in an inert solvent such as dimethylformamide.

Included within the scope is protecting group interchange and use of optional protecting groups within Ar¹, Ar², R¹, R², R³, R⁴ and X for example when X is NH, preferably a protecting group is used. 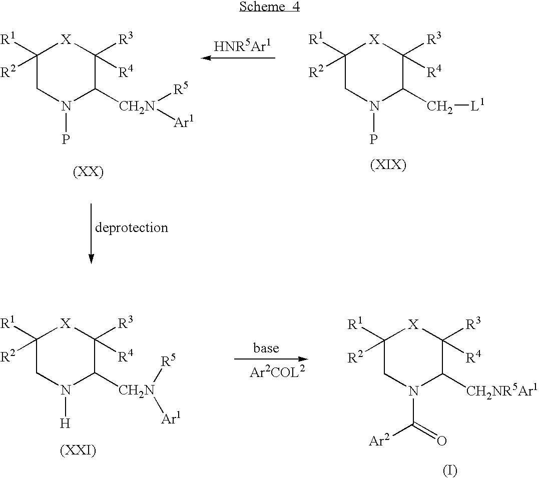
wherein Ar¹, Ar², R¹ to R⁵ and X are as defined for formula (I), L¹ and L² are leaving groups, and P is a protecting group.

Examples of suitable leaving groups L¹ include halogen, hydroxy, OSO₂Me, OSO₂(4-tolyl). The reaction of (XX) with HNR⁵Ar¹ preferably proceeds in an inert solvent such as N,N-dimethylformamide in the presence of a base such as triethylamine, sodium hydride or potassium t-butoxide.

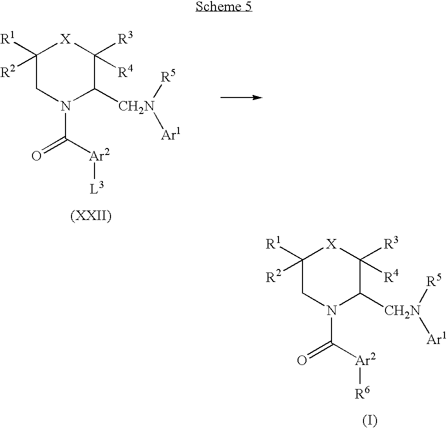

wherein Ar¹, Ar², R¹ to R⁶ and X are as defined for compounds of formula (I). L³ is a leaving group.

The compounds of formula (I) may be prepared singly or as compound libraries comprising at least 2, e.g. 5 to 1000, preferably 10 to 100 compounds of formula (I). Compound libraries may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I), or pharmaceutically acceptable derivatives thereof.

Pharmaceutically acceptable salts may be prepared conventionally by reaction with the appropriate acid or acid derivative.

The compounds of formula (I) and their pharmaceutically acceptable derivatives are useful for the treatment of diseases or disorders where an antagonist of a human Orexin receptor is required such as obesity and diabetes; prolactinoma; hypoprolactinemia; hypothalamic disorders of growth hormone deficiency; idiopathic growth hormone deficiency; Cushings syndrome/disease; hypothalamic-adrenal dysfunction; dwarfism; sleep disorders; sleep apnea; narcolepsy; insomnia; parasomnia; jet-lag syndrome; sleep disturbances associated with diseases such as neurological disorders, neuropathic pain and restless leg syndrome; heart and lung diseases; depression; anxiety; addictions; obsessive compulsive disorder; affective neurosis/disorder; depressive neurosis/disorder; anxiety neurosis; dysthymic disorder; behaviour disorder; mood disorder; sexual dysfunction; psychosexual dysfunction; sex disorder; sexual disorder; schizophrenia; manic depression; delerium; dementia; bulimia and hypopituitarism. Additionally the compounds of formula (I) and pharmaceutically acceptable derivatives are useful for the treatment of stroke, particularly ischemic or haemorrhagic and/or in blocking an emetic response i.e. nausea and vomiting.

The compounds of formula (I) and their pharmaceutically acceptable derivatives are particularly useful for the treatment of obesity, including obesity associated with Type 2 diabetes, and sleep disorders. Additionally the compounds of formula (I) and pharmaceutically acceptable derivatives are useful for the treatment of stroke, particularly ischemic or haemorrhagic and/or in blocking an emetic response i.e. nausea and vomiting.

Other diseases or disorders which may be treated in accordance with the invention include disturbed biological and circadian rhythms; adrenohypophysis disease; hypophysis disease; hypophysis tumor/adenoma; adrenohypophysis hypofunction; functional or psychogenic amenorrhea; adrenohypophysis hyperfunction; migraine; hyperalgesia; pain; enhanced or exaggerated sensitivity to pain such as hyperalgesia, causalgia and allodynia; acute pain; burn pain; atypical facial pain; neuropathic pain; back pain; complex regional pain syndromes I and II; arthritic pain; sports injury pain; pain related to infection e.g. HIV, post-polio syndrome and post-herpetic neuralgia; phantom limb pain; labour pain; cancer pain; post-chemotherapy pain; post-stroke pain; post-operative pain; neuralgia; and tolerance to narcotics or withdrawal from narcotics.

The invention also provides a method of treating or preventing diseases or disorders where an antagonist of a human Orexin receptor is required, which comprises administering to a subject in need thereof an effective amount of a compound of formula (I), or a pharmaceutically acceptable derivative thereof.

The invention also provides a compound of formula (I), or a pharmaceutically acceptable derivative thereof, for use in the treatment or prophylaxis of diseases or disorders where an antagonist of a human Orexin receptor is required.

The invention also provides the use of a compound of formula (I), or a pharmaceutically acceptable derivative thereof, in the manufacture of a medicament for the treatment or prophylaxis of diseases or disorders where an antagonist of a human Orexin receptor is required.

For use in therapy the compounds of the invention are usually administered as a pharmaceutical composition. The invention also provides a pharmaceutical composition comprising a compound of formula (I), or a pharmaceutically acceptable derivative thereof, and a pharmaceutically acceptable carrier.

The compounds of formula (I) and their pharmaceutically acceptable derivatives may be administered by any convenient method, e.g. by oral, parenteral, buccal, sublingual, nasal, rectal or transdermal administration, and the pharmaceutical compositions adapted accordingly.

The compounds of formula (I) and their pharmaceutically acceptable derivatives which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochloro-hydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

The dose of the compound of formula (I), or a pharmaceutically acceptable derivative thereof, used in the treatment or prophylaxis of the abovementioned disorders or diseases will vary in the usual way with the particular disorder or disease being treated, the weight of the subject and other similar factors. However, as a general rule, suitable unit doses may be 0.05 to 1000 mg, more suitably 0.05 to 500 mg. Unit doses may be administered more than once a day for example two or three times a day, so that the total daily dosage is in the range of about 0.01 to 100 mg/kg; and such therapy may extend for a number of weeks or months. In the case of pharmaceutically acceptable derivatives the above figures are calculated as the parent compound of formula (I).

No toxicological effects are indicated/expected when a compound of formula (I) is administered in the above mentioned dosage range.

Human Orexin-A has the amino acid sequence:

```
pyroGlu Pro Leu Pro Asp Cys Cys Arg Gln Lys Thr
  1 5 10
Cys Ser Cys Arg Leu
          15
Tyr Glu Leu Leu His Gly Ala Gly Asn His Ala Ala
                        20 25
Gly Ile Leu Thr
  30
```

Leu-NH₂

Orexin-A can be employed in screening procedures for compounds which inhibit the ligand's activation of the orexin-1 receptor.

In general, such screening procedures involve providing appropriate cells which express the orexin-1 receptor on their surface. Such cells include cells from mammals, yeast, Drosophila or E. coli. In particular, a polynucleotide encoding the orexin-1 receptor is used to transfect cells to express the receptor. The expressed receptor is then contacted with a test compound and an orexin-1 receptor ligand to observe inhibition of a functional response. One such screening procedure involves the use of melanophores which are transfected to express the orexin-1 receptor, as described in WO 92/01810.

Another screening procedure involves introducing RNA encoding the orexin-1 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 receptor ligand to cells which have the receptor on their surface. This method involves transfecting a eukaryotic cell with DNA encoding the orexin-1 receptor such that the cell expresses the receptor on its surface and contacting the cell or cell membrane preparation with a compound in the presence of a labelled form of an orexin-1 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilisation of intracellular calcium ions, or other ions, by affecting the interaction of an orexin-1 receptor ligand with the orexin-1 receptor.

All publications, including but not limited to patents and patent applications, cited in this specification are herein incorporated by reference as if each individual publication were specifically and individually indicated to be incorporated by reference herein as though fully set forth.

The following Examples illustrate the preparation of pharmacologically active compounds of the invention. The Descriptions D1-D14 illustrate the preparation of intermediates to compounds of the invention.

### Description 1: (RS)—C-(3,3-Dimethyl-piperidin-2-yl)-methylamine

A 1M solution of lithium aluminium hydride in tetrahydrofuran (114 ml) was added dropwise to a stirred solution of (RS)-3,3-dimethyl-piperidine-2-carbonitrile [Martens et al, J. Chem. Soc., Perk Trans 1, 2001, 508-13] (15.7 g, 0.114 mol) at room temperature under argon. The resultant mixture was stirred at room temperature for 0.5 h then heated at reflux for 1 h. The mixture was cooled to room temperature and chilled in ice as water (3.5 ml), 20% sodium hydroxide (1.54 ml) and water (15 ml) were added dropwise sequentially with stirring. After 0.5 h, anhydrous sodium sulphate was added, stirring continued for 0.5 h and the mixture filtered and solids washed with diethyl ether. Combined filtrate and washings were evaporated in vacuo to afford the title compound as a pale orange oil (12.7 g, 79%). Mass spectrurn (APt): Found 143 (MH⁺). C₈H₁₈N₂ requires 142.

### Description 2: (RS)-(5-Bromo-pyrimidin-2-yl)-(3,3-dimethyl-piperidin-2-ylmethyl)-amine

A mixture of (RS)—C-(3,3-dimethyl-piperidin-2-yl)-methylamine (D1) (7 g, 0.049 mol), 5-bromo-2-chloropyrimidine (9.52 g, 0.049 mol), diisopropylethylamine (26.4 ml, 0.147 mol) and potassium carbonate (13.6 g, 0.099 mol) in xylene (250 ml) was heated at 120° C. under argon for 20 h. On cooling the mixture was filtered, the filter cake washed with ethyl acetate and the combined filtrate and washings evaporated in vacuo. The residue was chromatographed on silica gel eluting with 0-10% methanol in ethyl acetate gradient then a 10% methanol in ethyl acetate mixture containing 2-4% 0.880 ammonia to afford the title compound as a pale orange solid (4.5 g, 31%). Mass spectrum (Electrospray LC/MS): Found 299 (MH⁺). C₁₂H₁₉⁷⁹BrN₄ requires 298.

The racemic product of Description 2 was separated into its individual enantiomers using the following procedure. Racemate (0.94 g) was dissolved in ethanol to a concentration of 100 mgml⁻¹. A 2 ml aliquot of this solution was applied to a Chiralpak AD (250 mm×20 mm i.d.) chromatography column. Elution with ethanol at a flow rate of 17 mlmin⁻¹ using U.V. detection at 215 nm afforded the individual enantiomers. Repeat injection of 2 ml aliquots, pooling of relevant fractions and evaporation of the pooled fractions in vacuo afforded the following:—

Description (2a): Faster running enantiomer (0.29 g). Mass spectrum (Electrospray LC/MS):

Found 299 (MH⁺). C₁₂H₁₉⁷⁹BrN₄ requires 298. Enantiomeric purity 99.9% e.e. [α]_{D}=+55.1° (c=1, CHCl₃, at 29° C.)

Description (2b): Slower running enantiomer (0.29 g). Mass spectrum (Electrospray LC/MS):

Found 299 (MH⁺). C₁₂H₁₉⁷⁹BrN₄ requires 298. Enantiomeric purity 99.9% e.e. [α]_{D}=51.2° (c=1, CHCl₃, at 28° C.)

### Description 3: (RS)-(5-Bromo-pyridin-2-yl)-(3,3-dimethyl-piperidin-2-ylmethyl)-amine

A mixture of (RS)-C-(3,3-dimethyl-piperidin-2-yl)methylamine (D1) (0.2 g, 1.41 mmol), 5-bromo-2-fluoro-pyridine (0.248 g, 1.41 mmol), diisopropylethylamine (0.76 ml, 4.23 mmol) and potassium carbonate (0.389 g, 2.82 mmol) in anhydrous dimethylformamide (5 ml) was heated at 100° C. under argon for 20 h. The reaction mixture was cooled, evaporated in vacuo and the residue chromatographed on silica gel eluting with 0-10% methanol in ethyl acetate gradient then a 10% methanol in ethyl acetate mixture containing 24% 0.880 ammonia to afford the title compound as a pale orange oil (0.28 g, 67%). Mass spectrum (Electrospray LC/MS): Found 298 (MH⁺). C₁₃H₂₀⁷⁹BrN₃ requires 297.

The racemic product of Description 3 was separated into its individual enantiomers using the following procedure. Racemate (3.2 g) was dissolved in ethanol to a concentration of 20 mgml⁻¹. A 1 ml aliquot of this solution was applied to a Chiralpak AD (250 mm×20 mm i.d.) chromatography column. Elution with ethanol at a flow rate of 17 mlmin⁻¹ using U.V. detection at 215 nm afforded the individual enantiomers. Repeat injection of 1 ml aliquots, pooling of relevant fractions and evaporation of the pooled fractions in vacuo afforded the following:—

Description (3a): Faster running enantiomer (0.45 g). Mass spectrum (Electrospray LC/MS):

Found 298 (MH⁺). C₁₃H₂₀⁷⁹BrN₃ requires 297. Enantiomeric purity 96.4% e.e. [α]_{D}=+47.5° (c=1, CHCl₃, at 30° C.)

Description (3b): Slower running enantiomer (0.45 g). Mass spectrum (Electrospray LC/MS):

Found 298 (MH⁺). C₁₃H₂₀⁷⁹BrN₃ requires 297. Enantiomeric purity 94.8% e.e. [α]_{D}=−46.8° (c=1, CHCl₃, at 28° C.)

### Description 4: (RS)-5,5-Dimethyl-piperidine-2-carboxylic acid

A mixture of 3-chloro-6,6-dimethyl-azepan-2-one [EP0447704 A1] (18 g, 0.103 mol) and barium hydroxide octahydrate (40.6 g, 0.129 mol) in water (600 ml) was heated at reflux for 20 h. On cooling, ammonium sulfate (17.13 g, 0.129 mol) was added with stirring. The mixture was filtered through kieselguhr and the filtrate evaporated in vacuo. The residue was mixed with toluene and the mixture evaporated in vacuo to afford the title compound as a colourless solid (20 g) which was used without further purification. ¹H NMR (D₂O) δ 1.00 (6H, m), 1.40-1.70 (2H, m), 1.75-1.90 (1H, m), 2.05-2.15 (1H, m), 2.75-2.85 (1H, m), 3.03 (1H, m), 3.40-3.55 (1H, m).

### Description 5: (RS)-5,5-Dimethyl-piperidine-1,2-dicarboxylic acid 1-tert-butyl ester

To a solution of D4 (20 g) and triethylamine (19.5 ml, 0.14 mol) in dioxan (800 ml) and water (140 ml) was added di-t-butyl dicarbonate (30.6 g, 0.14 mol). The resultant mixture was stirred at room temperature for 48 h. then evaporated in vacuo. The residue was partitioned between 1N sodium hydroxide and ethyl acetate (500 ml). The organic layer was extracted with 1N sodium hydroxide (2×100 ml). Combined aqueous layers were adjusted to pH6 with 5N hydrochloric acid and extracted with dichloromethane (3×250 ml). Combined extracts were dried (Na₂SO₄) and evaporated in vacuo to afford the title compound as a pale yellow gum (10.3 g). NMR (CDCl₃) inter alia δ: 0.85-0.95 (6H, m), 1.10-1.30 (2H, m), 1.45 (9H, m), 1.87 (1H, m), 2.10 (1H, m), 2.75-2.95 (1H, m), 3.50-3.65 (1H, m), 4.60-4.90 (1H, m), 7.20-8.90 (1H, br s).

### Description 6: (RS)-2-Carbamoyl-5,5-dimethyl-piperidine-1-carboxylic acid tert-butyl ester

A mixture of (RS)-5,5-dimethyl-piperidine-1,2-dicarboxylic acid 1-tert butyl ester (D5) (9.5 g, 37 mmol), 1-(3-dimethylaminopropyl)-3-ethylcarbodiimide hydrochloride (10.63 g, 55 mol), 1-hydroxybenzotriazole (8.5 g, 55 mmol), diisopropylethylamine (26 ml, 148 mmol) and ammonium chloride (3.96 g, 74 mmol) in dimethylformamide (100 ml) was stirred at room temperature for 20 h. then concentrated in vacuo. The residue was partitioned between water (500 ml) and ethyl acetate (500 ml) and the aqueous layer extracted with ethyl acetate (3×200 ml). Combined organics were washed with water (3×300 ml), saturated sodium hydrogen carbonate(300 ml), 0.5N hydrochloric acid (500 ml) and brine (250 ml), dried (Na₂SO₄) and evaporated in vacuo. The residue was chromatographed on silica gel eluting with 0-50% ethyl acetate in pentane gradient to afford the title compound as a colourless solid (5.3 g, 54%). NMR (CDCl₃) δ: 0.88 (3H, s), 0.91 (3H, s), 1.20-1.50 (2H, m), 1.48 (9H, s), 1.75 (1H, m), 2.17 (1H, m), 2.57 (1H, br m), 3.40-3.85 (1H, br m), 4.83 (1H, br m), 5.58 (1H, br s), 6.06 (1H, br s).

### Description 7: (RS)-5,5Dimethyl-piperidine-2-carboxylic acid amide

A solution of (RS)-2-carbamoyl-5,5-dimethyl-piperidine-1-carboxylic acid tert-butyl ester (D6) (5.2 g, 0.02 mol) in dichloromethane (50 ml) and trifluoroacetic acid (15 ml) was heated at 40° C. for 0.5 h. The reaction mixture was evaporated in vacuo and the residue partitioned between dichloromethane (100 ml) and 1N sodium hydroxide (100 ml). The aqueous layer was extracted with dichloromethane (3×100 ml) and the combined organics dried (Na₂SO₄) and evaporated in vacuo to give the title compound as a colourless solid (3 g, 95%). NMR (CDCl₃) δ: 0.89 (3H, s), 0.97 (3H, s), 1.20-1.35 (1H, m), 1.40-1.55 (1H, m), 1.60-1.75 (2H, m), 1.80-1.90 (1H, m), 2.45 (1H, m), 2.60 (1H, m), 3.15 (1H, m), 5.5 (1H, br s), 6.75 (1H, br s).

### Description 8: (RS)-1-Benzyl-5,5-dimethyl-piperidine-2-carboxylic acid amide

A solution of (RS)-5,5-dimethyl-piperidine-2-carboxylic acid amide (D7) (3 g, 19.2 mmol) and benzaldehyde (2.15 ml, 21.2 mmol) in 1,2-dichloroethane (120 ml) was stirred at room temperature for 1.5 h under argon prior to addition of sodium triacetoxyborohydride (6.08 g, 28.7 mmol) in one portion. The reaction mixture was stirred at room temperature for 24 h, diluted with dichloromethane (120 ml) and washed with saturated sodium hydrogen carbonate. The organic layer was dried (Na₂SO₄) and evaporated in vacuo. The residue was chromatographed on silica gel eluting with 0-50% ethyl acetate in pentane gradient to afford the title compound as a colourless solid (4.24 g, 90%). Mass spectrum (APIf): Found 247 (MH⁺). C₁₅H₂₂N₂O requires 246.

### Description 9: (RS)—C-(1-Benzyl-5,5-dimethyl-piperidin-2-yl)-methylamine

A 1M solution of lithium aluminium hydride in tetrahydrofuran (20.7 ml) was added dropwise over 0.15 h to a stirred solution of (RS)-1-benzyl-5,5-dimethyl-piperidine-2-carboxylic acid amide (D8) (4.22 g, 17.2 mmol) and the resultant mixture stirred at room temperature for 0.5 h then at reflux for a further 3 h. On cooling, water (3.7 ml), 2N sodium hydroxide (4.12 ml) and water (3.7 ml) were added dropwise sequentially followed after 0.1 h by anhydrous sodium sulphate. The mixture was filtered, the solids washed with tetrahydrofuran and the filtrates and washings combined and evaporated in vacuo to afford the title compound as a colourless solid (4 g, 100%). NMR (CDCl₃) δ: 0.79 (3H, s), 0.97 (3H, s), 1.10-1.60 (5H, m), 1.65-1.90 (2H, m), 2.15 (1H, m), 2.30-2.45 (1H, m), 2.65-2.75 (1H, m), 3.0-3.15 (2H, m), 4.07 (1H, m), 7.10-7.35 (5H, m).

### Description 10: (RS)—N-(1-Benzyl-5,5-dimethyl-piperidin-2-ylmethyl)-2,2,2-trifluoroacetamide

Trifluoroacetic anhydride (2.92 ml, 20.6 mmol) was added dropwise to a stirred solution of (RS)—C-(1-benzyl-5,5-dimethyl-piperidin-2-yl)-methylamine (D9) (3.98 g, 17.2 mmol) and triethylamine (3.35 ml, 24 mmol) in anhydrous dichloromethane (100 ml) at 0° C. under argon. The resultant mixture was allowed to warm to room temperature, stirred for 20 h, and washed with saturated sodium hydrogen carbonate. The organic layer was dried (Na₂SO₄) and evaporated in vacuo. The residue was chromatographed on silica gel eluting with 0-50% ethyl acetate in pentane gradient to yield the title compound as a colourless solid (3.85 g, 66%). Mass spectrum (Electrospray LC/MS):

Found 329 (MH⁺). C₁₇H₂₃F₃N₂O requires 328.

### Description 11: (RS)-5,5-Dimethyl-2-[(2,2,2-trifluoro-ethanoylamino)-methyl]-piperidine-1-carboxylic acid tert-butyl ester

A solution of (RS)—N-(1-benzyl-5,5-dimethyl-piperidin-2-ylmethyl)-2,2,2-trifluoroacetamide (D10) (4.75 g, 14.5 mmol) and di-tert-butyl dicarbonate (3.8 g, 17.4 mmol) in ethanol (100 ml) was hydrogenated at atmospheric pressure and room temperature in the presence of 10% palladium on carbon (1 g, 54% paste with water) for 70 h. The mixture was filtered through kieselguhr, the filtrate evaporated in vacuo and the residue chromatographed on silica gel eluting with 0-10% ethyl acetate in pentane gradient to give the title compound as a colourless solid (4.57 g, 93%). Mass spectrum

(API⁺): Found 339 (MH⁺). C₁₅H₂₅F₃N₂O₃ requires 338.

### Description 12: (RS)-2-Aminomethyl-5,5-dimethyl-piperidine-1-carboxylic acid tert butyl ester

A mixture of (RS)-5,5-dimethyl-2-[(2,2,2-trifluoro-ethanoylamino)-methyl]-piperidine-1-carboxylic acid tert butyl ester (D11) (4.56 g, 12.6 mmol) and sodium carbonate (9.4 g, 89 mmol) in methanol (300 ml) and water (100 ml) were heated at reflux for 2.5 h under argon. Potassium carbonate (9.4 g, 68 mmol) was added and reflux continued for 1 h. The reaction mixture was cooled, evaporated in vacuo and the residue partitioned between water (100 ml) and dichloromethane (300 ml). The aqueous layer was extracted with dichloromethane (2×100 ml) and the combined organics dried (Na₂SO₄) and evaporated in vacuo to yield the title compound as a pale orange oil (3.2 g, 98%). NMR (CDCl₃) δ: 0.89 (3H, s), 0.91 (3H, s), 1.20-1.80 (5H, m), 1.46 (9H, s), 1.82 (1H, m), 2.50 (1H, m), 2.62 (1H, m), 2.90 (1H, m), 3.60 (1H, br s), 4.15 (1H, br s).

### Description 13: (RS)-2-[5-Bromo-pyrimidin-2-ylamino)-methyl]-5,5-dimethyl-piperidine-1-carboxylic acid tert-butyl ester

A mixture of (RS)-2-aminomethyl-5,5-dimethyl-piperidine-1-carboxylic acid tert-butyl ester (2.7 g, 11.2 mmol), 5-bromo-2-chloropyrimidine (2.04 g, 11.2 mmol), potassium carbonate (3.1 g, 22.5 mmol) and diisopropylethylamine (5.9 ml, 33.5 mmol) in xylene (100 ml) was heated at 130° C. for 20 h under argon. The reaction mixture was cooled, filtered and the solid washed with ethyl acetate. Combined filtrates and washings were evaporated in vacuo and the residue chromatographed on silica gel eluting with 0-20% ethyl acetate in pentane gradient to give the title compound as a colourless solid (3.6 g, 80%). NMR (CDCl₃) δ: 0.91 (6H, s), 1.27 (1H, m), 1.30-1.55 (2H, m), 1.40 (9H, s), 1.88 (1H, m), 2.59 (1H, m), 3.33 (1H, m), 3.40-3.75 (2H, m), 4.96 (1H, br s), 5.30 (1H, br s), 8.25 (2H, s).

### Description 14: (RS)-(5-Bromo-pyrimidin-2-yl)-(5,5-dimethyl-piperidin-2-ylmethyl)-amine

The title compound was prepared from (RS)-2-[5-bromo-pyrimidin-2-ylamino)-methyl]-5,5-dimethyl-piperidin-1-carboxylic acid tert-butyl ester (D13) (3.6 g, 9.0 mmol) using the method of description D7 as a pale orange solid (2.6 g, 97%). NMR (CDCl₃) δ: 0.85 (3H, s), 0.98 (3H, s), 1.20-1.60 (5H, m), 2.42 (1H, m), 2.55-2.70 (2H, m), 3.25 (1H, m), 3.46 (1H, m), 5.64 (1H, br s), 8.26 (2H, s).

The racemic product of Description 14 was separated into its individual enantiomers using the following procedure. Racemate (2.5 g) was dissolved in ethanol to a concentration of 200 mgml⁻¹. A 1 ml aliquot of this solution was applied to a Chiralpak AD (200 mm×50 mm i.d.) chromatography column. Elution with ethanol containing 0.1% triethylamine at a flow rate of 50 mlmin⁻¹ using U.V. detection at 230 nm afforded the individual enantiomers. Repeat injection of 1 ml aliquots, pooling of relevant fractions and evaporation of the pooled fractions in vacuo afforded the following:—

Description (14a): Faster running enantiomer (1.07 g). Mass spectrum (Electrospray LC/MS):

Found 299 (MH⁺). C₁₂H₁₉⁷⁹BrN₄ requires 298. Enantiomeric purity 99% e.e. [α]_{D}=−21.7° (c=1, CHCl₃, at 29° C.)

Description (14b): Slower running enantiomer (0.975 g). Mass spectrum (Electrospray LC/MS):

Found 299 (MH⁺). C₁₂H₁₉⁷⁹BrN₄ requires 298. Enantiomeric purity 98% e.e. [α]_{D}=+16.5° (c=1, CHCl₃, at 29° C.)

### EXAMPLE 1

### (RS)-{2-1(5-Bromo-pyrimidin-2-ylamino)-methyl]-3,3-dimethyl-piperidin-1-yl)-1-[5-(4-fluoro-phenyl)-2-methyl-thiazol-4yl]-methanone

A solution of 5-(4-fluoro-phenyl)-2-methyl-thiazole-4-carbonyl chloride (0.094 g, 0.37 mmol) in dichloromethane (1 ml) was added dropwise to a stirred solution of (RS)-5-bromo-pyrimidin-2-yl)-(3,3-dimethyl-piperidin-2-ylmethyl)-amine (D2) (0.1 g, 0.33 mmol) and triethylamine (0.1 ml, 0.74 mmol) in dichloromethane (3 ml). After 2.5 h the reaction mixture was washed with saturated sodium hydrogen carbonate (8 ml) and the organic layer applied to a 10 g silica gel column. Elution with 0-100% ethyl acetate in hexane gradient afforded the title compound as a colourless solid (0.135 g, 78%). Mass spectrum (Electrospray LC/MS) Found 518 (MH⁺). C23H₂₅⁷⁹BrFN₅OS requires 517.

### EXAMPLE 2

### (RS)-1-(2-[(5-Bromo-pyrimidin-2-ylamino)-methyl]-3,3-dimethyl-piperidin-1-yl}-1-quinolin-8yl-methanone

A mixture of (RS)-(5-bromo-pyrimidin-2-yl)-(3,3-dimethyl-piperidin-2-ylmethyl)-amine (D2) (0.08 g, 0.27 mmol), quinoline-8-carboxylic acid (0.046 g, 0.27 mmol), O-(7-azabenzotriazol-1-yl)-N,N,N¹,N¹-tetramethyluronium hexafluorophosphate (HATU) (0.102 g, 0.27 mmol) and diisopropylethylamine (0.14 ml, 0.81 mmol) in anhydrous dimethylformamide (6 ml) was stirred at room temperature for 24 h. The reaction mixture was evaporated in vacuo and the residue dissolved in ethyl acetate and washed with water. The organic layer was dried (Na₂SO₄) and evaporated in vacuo. The residue was chromatographed on silica gel using 0-100% ethyl acetate in pentane gradient then 0-10% methanol in ethyl acetate gradient to afford the title compound as a colourless solid (0.039 g, 32%). Mass spectrum (Electrospray LC/MS): Found 454 (MH⁺). C₂₂H₂₄⁷⁹BrN₅O requires 453.

### EXAMPLE 3

### 1-{2-[(5-Bromo-pyrimidin-2-ylamino)-methyl]-3,3-dimethyl-piperidin-1-yl}-1-(2-methyl-quinolin-5-yl)-methanone

A mixture of (+)-(5-bromo-pyrimidin-2-yl)-(3,3-dimethyl-piperidin-2-ylmethyl)-amine (D2a) (0.075 g, 0.25 mmol), 2-methyl-quinoline-5-carboxylic acid (0.047 g, 0.25 mmol), HATU (0.096 g, 0.25 mmol) and diisopropylethylamine (0.13 ml, 0.75 mmol) in anhydrous dimethylformamide (3.5 ml) was stirred at room temperature for 24 h. The reaction mixture was evaporated in vacuo and the residue dissolved in ethyl acetate and washed with water. The organic layer was dried (Na₂SO₄) and evaporated in vacuo. The residue was chromatographed on silica gel using 0-100% ethyl acetate in pentane gradient then 0-10% methanol in ethyl acetate gradient to afford the title compound as a colourless solid (0.06 g, 53%). Mass spectrum (Electrospray LC/MS): Found 468 (MH⁺). C₂₃H₂₆⁷⁹BrN₅O requires 467. The absolute stereochemistry of Example 3 is undefined.

### EXAMPLE 4

### (RS)-1-[(5-Bromo-pyrimidin-2-ylamino)-methyl]-5,5-dimethyl-piperidin-1-yl}-1-(2-methyl-quinolin-5-yl)-methanone

A mixture of (RS)-5-(bromo-pyrimidin-2-yl)-(5,5-dimethyl-piperidin-2-ylmethyl)-amine (D14) (0.1 g, 0.33 mmol), 2-methyl-quinoline-5-carboxylic acid (0.068 g, 0.36 mmol), HATU (0.125 g, 0.33 mmol) and diisopropylethylamine (0.18 ml, 0.99 mmol) in anhydrous dimethylformamide (5 ml) was stirred at room temperature for 24 h. The reaction mixture was evaporated in vacuo and the residue dissolved in ethyl acetate and washed with water. The organic layer was dried (Na₂SO₄) and evaporated in vacuo. The residue was chromatographed on silica gel using 0-100% ethyl acetate in pentane gradient then 0-10% methanol in ethyl acetate gradient to afford the title compound as a colourless solid (0.103 g, 66%). Mass spectrum (Electrospray LC/MS): Found 468 (MH⁺). C₂₃H₂₆⁷⁹BrN₅O requires 467.

### EXAMPLE 5

### 1-{2-[(5Bromo-pyrimidin-2-ylamino)-methyl]-5,5-dimethyl-piperidin-1-yl}-1-(2-methyl-quinolin-5-yl)-methanone

A mixture of (+)-5-(bromo-pyrimidin-2-yl)-(5,5-dimethyl-piperidin-2-ylmethyl)-amine (D14b) (0.12 g, 0.4 mmol), 2-methyl-quinoline-5-carboxylic acid (0.075 g, 0.4 mmol), 1-[3-(dimethylamino)propyl]-3-ethylcarbodiimide hydrochloride (EDC) (0.077 g, 0.4 mmol) and 1-hydroxybenzotriazole hydrate (HOBt) (0.01 g, 0.07 mmol) in dichloromethane (4 ml) was shaken at room temperature for 20 h. The reaction mixture was washed with saturated aqueous sodium hydrogen carbonate (8 ml) and the organic layer applied to a pre-packed 10 g silica column. Elution with 0-100% ethyl acetate in pentane gradient then 0-10% methanol in ethyl acetate gradient gave the title compound as a colourless solid (0.062 g, 33%). Mass spectrum (Electrospray LC/MS):

Found 468 (M⁺). C₂₃H₂₆⁷⁹BrN₅O requires 467.

### EXAMPLE 6

### (RS)-1-{2-[(5-Bromo-pyridin-2-ylamino)-methyl]-3,3-dimethyl-piperidin-1-yl}-1-(2,3-dimethyl-quinoxalin-5-yl)-methanone

A mixture of (RS)-(5-bromo-pyridin-2-yl)-(3,3-dimethyl-piperidin-2-ylmethyl)-amine (D3) (0.12 g, 0.4 mmol), 2,3-dimethyl-quinoxaline-5-carboxylic acid (0.082 g, 0.4 mmol), 1-[3-(dimethylamino)propyl]-3-ethylcarbodiimide hydrochloride (EDC) (0.077 g, 0.4 mmol) and 1-hydroxybenzotriazole hydrate (HOBt) (0.01 g, 0.07 mmol) in dichloromethane (4 ml) was shaken at room temperature for 20 h. The reaction mixture was washed with saturated aqueous sodium hydrogen carbonate (8 ml) and the organic layer applied to a pre-packed 10 g silica column. Elution with 0-100% ethyl acetate in pentane gradient then 0-10% methanol in ethyl acetate afforded a colourless solid. Mass directed purification on an ABZ⁺ C8 (100 mm×21 mm i.d.) chromatography column eluting with 0-95% acetonitrile in water containg 0.1% trifluoroacetic acid at a flow rate of 3 mlmin⁻¹ afforded the title compound as the trifluoroacetate salt (0.05 g, 21%). Mass spectrum (Electrospray LC/MS): Found 482 (MH⁺). C₂₄H₂₈⁷⁹BrN₅O requires 481.

### EXAMPLE 7

### (RS)-1-{2-[(5-Bromo-pyridin-2-ylamino)-methyl]-3,3-dimethyl-piperidin-1-yl}-1-(2-methyl-quinolin-5-yl)-methanone

A mixture of (RS)-5-bromo-pyridin-2-yl)-(3,3-dimethyl-piperidin-2-ylmethyl)-amine (D3) (0.1 g, 0.34 mmol), 2-methyl-quinoline-5-carboxylic acid (0.063 g, 0.34 mmol), 1-[3-(dimethylamino)propyl]-3-ethylcarbodiimide hydrochloride (EDC) (0.064 g, 0.34 mmol) and 1-hydroxybenzotriazole hydrate (HOBt) (0.01 g, 0.07 mmol) in dichloromethane (4 ml) was shaken at room temperature for 20 h. The reaction mixture was washed with saturated aqueous sodium hydrogen carbonate (8 ml) and the organic layer applied to a pre-packed 10 g silica column. Elution with 0-100% ethyl acetate in pentane gradient then 0-10% methanol in ethyl acetate gradient gave the title compound as a colourless solid (0.037 g, 23%). Mass spectrum (Electrospray LC/MS):

Found 467 (MH⁺) C₂₄H₂₇⁷⁹BrN₄O requires 466.

The compounds of the Examples below were prepared from the appropriate piperidine using similar processes to that described in Examples 1 to 7.

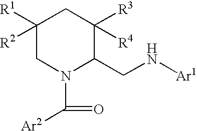

| | | | | | | | | Mass Spectrum |
|---|---|---|---|---|---|---|---|---|
| | | | | | | | | (Electrospray |
| Example | R¹ | R² | R³ | R⁴ | Ar¹ | Ar² | Amine | LC/MS) |
| 8 | H | H | Me | Me | 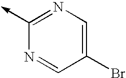 | 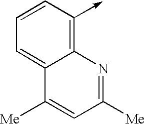 | D2 | Found 482 (MH⁺). C₂₄H₂₈^{79BrN}₅O requires 481. |
| 9 | H | H | Me | Me | 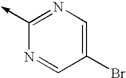 | 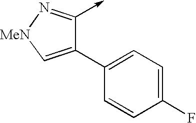 | D2 | Found 501 (MH⁺). C₂₃H₂₆^{79BrFN}₆O requires 500. |
| 10 | H | H | Me | Me | 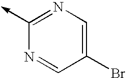 | 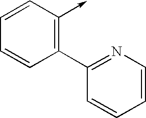 | D2 | Found 502 (MH⁺). C₂₄H₂₆^{79BrN}₅O requires 479. |
| 11 | H | H | Me | Me | 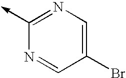 | 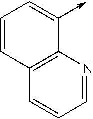 | D2a | Found 454 (MH⁺). C₂₂H₂₄^{79BrN}₅O requires 453. |
| 12 | H | H | Me | Me | 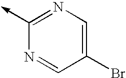 | 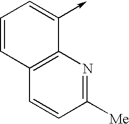 | D2a | Found 468 (MH⁺). C₂₃H₂₆^{79BrN}₅O requires 467. |
| 13 | H | H | Me | Me | 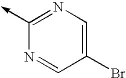 | 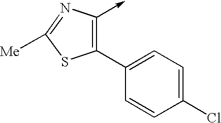 | D2a | Found 534 (MH⁺). C₂₃H₂₅⁷⁹Br³⁵ClN₅requires 533. |
| 14 | Me | Me | H | H | 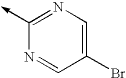 | 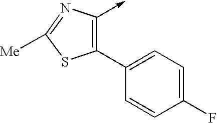 | D14 | Found 518 (MH⁺). C₂₃H₂₅⁷⁹BrFN₅OS requires 517. |
| 15 | Me | Me | H | H | 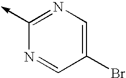 | 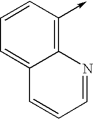 | D14 | Found 454 (MH⁺). C₂₂H₂₄⁷⁹BrN₄O requires 453. |
| 16 | H | H | Me | Me | 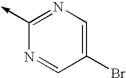 | 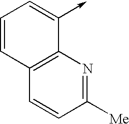 | D3 | Found 467 (MH⁺). C₂₄H₂₇⁷⁹BrN₄O requires 466. |
| 17 | H | H | Me | Me | 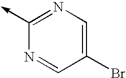 | 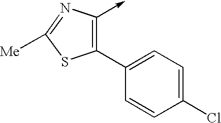 | D3 | Found 533 (MH⁺). C₂₄H₂₆⁷⁹Br³⁵ClN₄OS requires 532. |
| 18 | H | H | Me | Me | 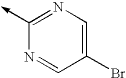 | 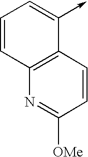 | D2a | Found 484 (MH⁺). C₂₃H₂₆⁷⁹BrN₅O₂requires 483. |
| 19 | H | H | Me | Me | 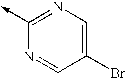 | 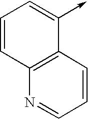 | D2a | Found 454 (MH⁺). C₂₂H₂₄⁷⁹BrN₅O requires 453. |
| 20 | H | H | Me | Me | 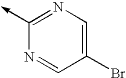 | 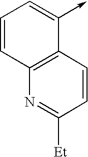 | D2a | Found 482 (MH⁺). C₂₄H₂₈⁷⁹BrN₅O requires 481. |
| 21 | H | H | Me | Me | 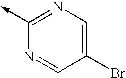 | 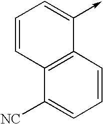 | D2a | Found 478 (MH⁺). C₂₄H₂₄⁷⁹BrN₅O requires 477. |
| 22 | H | H | Me | Me | 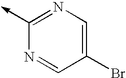 | 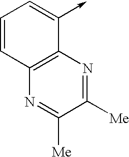 | D2a | Found 483 (MH⁺). C₂₃H₂₇⁷⁹BrN₆O requires 482. |
| 23 | H | H | Me | Me | 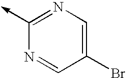 | 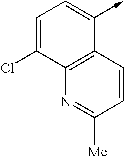 | D2a | Found 502 (MH⁺). C₂₃H₂₅⁷⁹Br³⁵ClN₅O requires 501. |
| 24 | H | H | Me | Me | 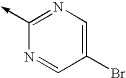 | 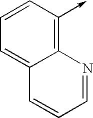 | D3 | Found 453 (MH⁺). C₂₃H₂₅⁷⁹BrN₄O requires 452. |
| 25 | H | H | Me | Me | 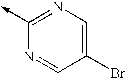 | 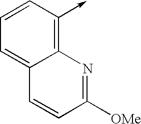 | D2a | Found 484 (MH⁺). C₂₃H₂₆⁷⁹BrN₅O2 requires 483. |
| 26 | H | H | Me | Me | 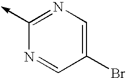 | 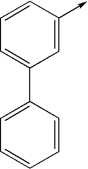 | D2a | Found 479 (MH⁺). C₂₅H₂₇⁷⁹BrN₄O requires 478. |
| 27 | H | H | Me | Me | 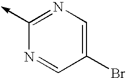 | 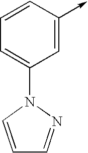 | D2a | Found 469 (MH⁺). C₂₂H₂₅⁷⁹BrN₆O requires 468. |
| 28 | H | H | Me | Me | 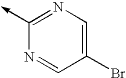 | 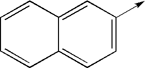 | D2a | Found 453 (MH⁺). C₂₃H₂₅⁷⁹BrN₄O requires 452. |
| 29 | H | H | Me | Me | 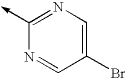 | 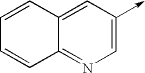 | D2a | Found 454 (MH⁺). C₂₂H₂₄⁷⁹BrN₅O requires 453. |
| 30 | H | H | Me | Me | 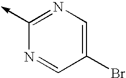 | 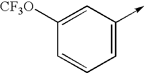 | D2a | Found 487 (MH⁺). C₂₀H₂₂⁷⁹BrF₃N₄O₂requires 486. |
| 31 | H | H | Me | Me | 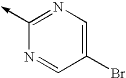 | 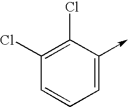 | D3a | Found 470 (MH⁺). C₂₀H₂₂⁷⁹Br³⁵Cl₂N₃O requires 469. |
| 32 | H | H | Me | Me | 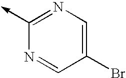 | 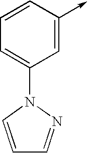 | D3a | Found 468 (MH⁺). C₂₃H₂₆⁷⁹BrN₅O requires 467. |
| 33 | H | H | Me | Me | 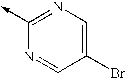 | 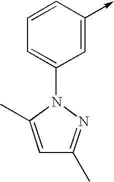 | D2a | Found 497 (MH⁺). C₂₄H₂₉⁷⁹BrN₆O requires 496. |
| 34 | H | H | Me | Me | 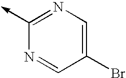 | 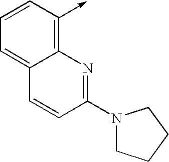 | D2a | Found 523 (MH⁺). C₂₆H₃₁⁷⁹BrN₆O requires 522. |
| 35 | Me | Me | H | H | 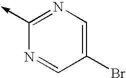 | 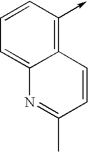 | D14b | Found 468 (MH⁺). C₂₃H₂₆⁷⁹BrN₅O requires 467. |
| 36 | H | H | Me | Me | 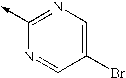 | 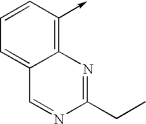 | D3a | Found 482 (MH⁺). C₂₄H₂₈⁷⁹BrN₅O requires 481. |
| 37 | H | H | Me | Me | 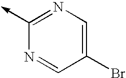 | 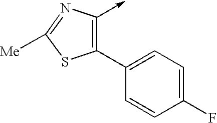 | D2a | Found 518 (MH⁺). C₂₃H₂₅⁷⁹BrFN₅OS requires 517. |
| 38 | H | H | Me | Me | 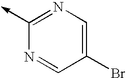 | 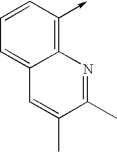 | D3a | Found 481 (MH⁺). C₂₅H₂₉⁷⁹BrN₄O requires 480. |
| 39 | H | H | Me | Me | 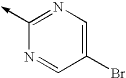 | 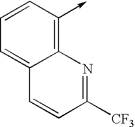 | D3a | Found 521 (MH⁺). C₂₄H₂₄⁷⁹BrF₃N₄O requires 520. |
| 40 | H | H | Me | Me | 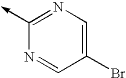 | 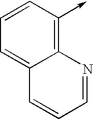 | D3a | Found 453 (MH⁺). C₂₃H₂₅⁷⁹BrN₄O requires 452. |
| 41 | H | H | Me | Me | 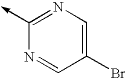 | 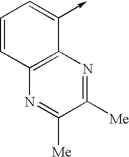 | D3a | Found 482 (MH⁺). C₂₄H₂₈⁷⁹BrN₅O requires 481. |
| 42 | H | H | Me | Me | 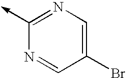 | 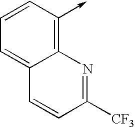 | D2a | Found 522 (MH⁺). C₂₃H₂₃⁷⁹BrF₃N₅O requires 521. |
| 43 | H | H | Me | Me | 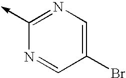 | 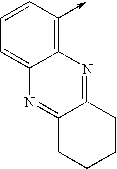 | D3a | Found 508 (MH⁺). C₂₆H₃₀⁷⁹BrN₅O requires 507. |

### EXAMPLE 44

### 1-{(S)-2-[(5-Bromo-pyrimidin-2-ylamino)-methyl]-3,3-dimethyl-piperidin-1-yl}-1-(2,3-dimethyl-quinolin-8yl)-methanone

To 2,3-dimethyl quinoline-8-carboxylic acid (0.5 g, 2.5 mmol) in dichloromethane (30 ml) was added oxalyl chloride (0.48 ml, 5.5 mmol) dropwise, followed by dimethylformamide (1 drop). The resulting solution was stirred at ambient temperature for 1 h. and then evaporated. The 2,3-dimethyl-8-quinolinecarbonyl chloride hydrochloride (0.64 g, 100%) thus obtained as a brown solid was used without purification. To a solution of (+)-(5-bromo-pyrimidin-2-yl)-(3,3-dimethyl-piperidin-2-ylmethyl)-amine (D2a) (0.1 g, 0.33 mmol) and triethylamine (0.2 ml, 1.44 mmol) in dichloromethane (5 ml) was added 2,3-dimethyl-8-quinolinecarbonyl chloride hydrochloride (0.087 g, 0.34 mmol). After 1 h at ambient temperature, the reaction mixture was washed with saturated aqueous sodium hydrogencarbonate and the organic layer applied to a silica gel column. Elution with an ethyl acetate-pentane gradient afforded the title product (0.5 g, 31%). Mass spectrum (Electrospray LC/MS): Found 482 (MH⁺). C₂₄H₂₈⁷⁹BrN₅O requires 481.

It is understood that the present invention covers all combinations of particular and preferred groups described herein above.

Determination of Orexin-1 Receptor Antagonist Activity

The orexin-1 receptor antagonist activity of the compounds of formula (I) was determined in accordance with the following experimental method.

Experimental Method

CHO-DG44 cells expressing the human orexin-1 receptor were grown in cell medium (MEM medium with Earl's salts) containing 2 mM L-Glutamine, 0.4 mg/mL G418 Sulphate from GIBCO BRL and 10% heat inactivated fetal calf serum from Gibco BRL. The cells were seeded at 20,000 cells/100 μl/well into 96-well black clear bottom sterile plates from Costar which had been pre-coated with 10 μg/well of poly-L-lysine from SIGMA. The seeded plates were incubated overnight at 37 C in 5% CO₂.

Agonists were prepared as 1 mM stocks in water:DMSO (1:1). EC50 values (the concentration required to produce 50% maximal response) were estimated using 11× half log unit dilutions (Biomek 2000, Beckman) in Tyrode's buffer containing probenecid (10 mM HEPES with 145 mM NaCl, 10 mM glucose, 2.5 mM KCl, 1.5 mM CaCl₂, 1.2 mM MgCl₂ and 2.5 mM probenecid; pH7.4). Antagonists were prepared as 10 mM stocks in DMSO (100%). Antagonist IC50 values (the concentration of compound needed to inhibit 50% of the agonist response) were determined against 3.0 nM human orexin-A using 11× half log unit dilutions in Tyrode's buffer containing 10% DMSO and probenecid.

On the day of assay 50 μl of cell medium containing probenecid (Sigma) and Fluo3AM (Texas Fluorescence Laboratories) was added (Quadra, Tomtec) to each well to give final concentrations of 2.5 mM and 4 μM, respectively. The 96-well plates were incubated for 60 min at 37 C in 5% CO2. The loading solution containing dye was then aspirated and cells were washed with 4×150 μl Tyrode's buffer containing probenecid and 0.1% gelatin (Denley Cell Wash). The volume of buffer left in each well was 125 μl. Antagonist or buffer (25 μl) was added (Quadra) the cell plates gently shaken and incubated at 37 C in 5% CO₂ for 30 minutes. Cell plates were then transferred to the Fluorescent Imaging Plate Reader (FLIPR, Molecular Devices) instrument. Prior to drug addition a single image of the cell plate was taken (signal test), to evaluate dye loading consistency. The run protocol used 60 images taken at 1 second intervals followed by a further 24 images at 5 second intervals. Agonists were added (by the FLIPR) after 20 seconds (during continuous reading). From each well, peak fluorescence was determined over the whole assay period and the mean of readings 1-19 inclusive was subtracted from this figure. The peak increase in fluorescence was plotted against compound concentration and iteratively curve fitted using a four parameter logistic fit (as described by Bowen and Jerman, TiPS, 1995, 16, 413-417) to generate a concentration effect value. Antagonist Kb values were calculated using the equation:
Kb=IC50/(1+([3/EC50])

where EC50 was the potency of human orexin-A determined in the assay (in nM terms) and IC50 is expressed in molar terms.

Compounds of Examples tested according to this method had pKb values in the range 7.0 to 9.7 at the human cloned orexin-1 receptor.

The orexin-2 receptor antagonist activity of the compounds of formula (I) was determined in accordance with the following experimental method.

Experimental Method

CHO-DG44 cells expressing the human orexin-2 receptor were grown in cell medium (MEM medium with Earl's salts) containing 2 mM L-Glutamine, 0.4 mg/mL G418 Sulphate from GIBCO BRL and 10% heat inactivated fetal calf serum from Gibco BRL. The cells were seeded at 20,000 cells/100 μl/well into 96-well black clear bottom sterile plates from Costar which had been pre-coated with 10 μg/well of poly-L-lysine from SIGMA. The seeded plates were incubated overnight at 37 C in 5% CO₂.

Agonists were prepared as 1 mM stocks in water:DMSO (1:1). EC50 values (the concentration required to produce 50% maximal response) were estimated using 11× half log unit dilutions (Biomek 2000, Beckman) in Tyrode's buffer containing probenecid (10 mM HEPES with 145 mM NaCl, 10 mM glucose, 2.5 mM KCl, 1.5 mM CaCl₂, 1.2 mM MgCl₂ and 2.5 mM probenecid; pH7.4). Antagonists were prepared as 10 mM stocks in DMSO (100%). Antagonist IC50 values (the concentration of compound needed to inhibit 50% of the agonist response) were determined against 10.0 nM human orexin-A using 11× half log unit dilutions in Tyrode's buffer containing 10% DMSO and probenecid.

On the day of assay 50 μl of cell medium containing probenecid (Sigma) and Fluo3AM (Texas Fluorescence Laboratories) was added (Quadra, Tomtec) to each well to give final concentrations of 2.5 mM and 4 μM, respectively. The 96-well plates were incubated for 60 min at 37 C in 5% CO₂. The loading solution containing dye was then aspirated and cells were washed with 4×150 μl Tyrode's buffer containing probenecid and 0.1% gelatin (Denley Cell Wash). The volume of buffer left in each well was 125 μl. Antagonist or buffer (25 μl) was added (Quadra) the cell plates gently shaken and incubated at 37 C in 5% CO₂ for 30 min. Cell plates were then transferred to the Fluorescent Imaging Plate Reader (FLIPR, Molecular Devices) instrument. Prior to drug addition a single image of the cell plate was taken (signal test), to evaluate dye loading consistency. The run protocol used 60 images taken at 1 second intervals followed by a further 24 images at 5 second intervals. Agonists were added (by the FLIPR) after 20 sec (during continuous reading). From each well, peak fluorescence was determined over the whole assay period and the mean of readings 1-19 inclusive was subtracted from this figure. The peak increase in fluorescence was plotted against compound concentration and iteratively curve fitted using a four parameter logistic fit (as described by Bowen and Jerman, TiPS, 1995, 16, 413-417) to generate a concentration effect value. Antagonist Kb values were calculated using the equation:
Kb=IC50/(1+([3/EC50])

where EC50 was the potency of human orexin-A determined in the assay (in nM terms) and IC50 is expressed in molar terms.

Compounds of Examples tested according to this method had pKb values in the range <6.3 to 8.2 at the human cloned orexin-2 receptor.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

1. A compound of formula (I): 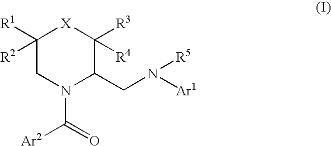
wherein:
X is O, CR⁷R⁸, NH or bond;
R¹ and R² are both hydrogen, both optionally substituted (C₁₋₄)alkyl, or are together with the carbon to which they are attached form a (C₃₋₆)cycloalkyl ring or a 4- to 6-membered heterocyclyl ring.
R³ and R⁴ are both hydrogen, both optionally substituted (C₁₋₄)alkyl, or are together with the carbon to which they are attached form a (C₃₋₆)cycloalkyl ring or a 4- to 6-membered heterocyclyl ring;
R⁷ and R⁸ are both hydrogen, both optionally substituted (C₁₋₄)alkyl, or are together with the carbon to which they are attached form a (C₃₋₆)cycloalkyl ring or a 4- to 6-membered heterocyclyl ring;
provided that one pair of R¹ and R², R³ and R⁴, R⁷ and R⁸ are both optionally substituted (C₁₋₄)alkyl, or are together with the carbon to which they are attached form a (C₃₋₆)cycloalkyl ring or a 4- to 6-membered heterocyclyl ring and the remaining groups are hydrogen;
R⁵ is hydrogen, optionally substituted (C₁₋₄) alkyl, or optionally substituted (C₁₋₄)alkylCO;
Ar¹ is an optionally substituted aryl, an optionally substituted mono or bicyclic heteroaryl group containing up to 3 heteroatoms selected from N, O and S;
Ar² represents phenyl or a 5- or 6-membered heterocyclyl group containing up to 3 heteroatoms selected from N, O and S, wherein the phenyl or heterocyclyl group is substituted by R⁶ and further optional substituents; or Ar² represents an optionally substituted bicyclic aromatic or bicyclic heteroaromatic group containing up to 4 heteroatoms selected from N, O and S;
R⁶ represents hydrogen, optionally substituted(C₁₋₄ )alkoxy, halo, cyano, optionally substituted(C₁₋₆)alkyl, optionally substituted phenyl, or an optionally substituted 5- or 6-membered heterocyclyl group containing up to 4 heteroatoms selected from N, O and S;
or a pharmaceutically acceptable salt thereof.

2. A compound according to claim 1 wherein X is CR⁷R⁸.

3. A compound according to claim 1 wherein R¹, R², R⁷ and R⁸ are hydrogen when R³ and R⁴ are methyl or R³, R⁴, R⁷ and R⁸ are hydrogen when R¹ and R² are methyl.

4. A compound according to claim 1 wherein Ar¹ is pyrimidinyl or pyridinyl.

5. A compound according to claim 4 wherein the pyrimidinyl or pyridinyl is substituted with halogen.

6. A compound according to claim 5 wherein the halogen is bromine.

7. A compound of formula (I) as defined in any one of Examples 1 to 44, or a pharmaceutically acceptable salt of any one thereof.

8. A pharmaceutical composition comprising a compound of formula (I) as defined in claim 1, or a pharmaceutically acceptable salt thereof, and a pharmaceutically acceptable carrier.

9. A method of treating or preventing diseases or disorders where an antagonist of a human orexin receptor is required, which comprises administering to a subject in need thereof an effective amount of a compound of formula (I) as defined in claim 1, or a pharmaceutically acceptable salt thereof.

10. The method according to claim 9, wherein said disease or disorder is obesity.

11. The method according to claim 9, wherein said disease or disorder is a sleep disorder.


SEQUENCE LISTING (1 sequence)

SEQ ID NO: 1 - protein, 35 aa, Homo sapien
EPLPDCCRQKTCSCRLYELLHGAGNHAAGILTLNH